(19) United States
(12) Patent Application Publication
(10) Pub. No.: US 2012/0010668 A1
(43) Pub. Date: Jan. 12, 2012

(54) EXPANDABLE SURGICAL IMPLANT

(75) Inventor: Daniel Andrew Shimko, Germantown, TN (US)

(73) Assignee: WARSAW ORTHOPEDIC, INC., Warsaw, IN (US)

(21) Appl. No.: 12/832,091

(22) Filed: Jul. 8, 2010

(51) Int. Cl. A61B 17/86

(52) U.S. Cl. 606/305; 606/313

(57) ABSTRACT

An implant includes an elongate body having at least two radially expandable portions in the vicinity of the distal end thereof. An expander is disposed at least partially within the axial bore of the body, the expander including a longitudinal shaft and a tip at the distal end of the shaft. The expander is longitudinally movable between a distal position wherein the tip is positioned distal to the distal end of the body and a proximal position wherein the expandable portions of the body are moved to a radially outward expanded configuration by a camming action of the tip.

### TECHNICAL FIELD

The present invention relates to implants that are surgically placed in bone to provide an anchoring means for a prosthesis or other device. In particular, the implant of the present invention is especially advantageous for use in spinal fixation.

### BACKGROUND

Implants are widely used in surgery for various purposes such as, for example, restoration of the jaw anatomy, ligament repair, spinal procedures, bone fixation, and the like. Implants need to be tightly secured to the bone tissue of a person into which they are placed for an extended period of time—typically for the life of the person. Implants are often secured by drilling a hole in the bone and then screwing the implant into the hole. After a period of time the bone tissue can grow into the implant to ensure permanent fixation. Implants can be used, for example, for anchoring a dental prosthesis such as an artificial tooth to the jawbone of a patient, for the repair of anterior cruciate ligament, repair of the knee, and for fusion of spinal vertebrae. Bone screws/anchors can be used to attach bone plates to join portions of broken bone.

The spinal column is a highly complex system of bones and connective tissues that provides support for the body and protects the delicate spinal column and nerves. The spinal column includes a series of vertebrae stacked one atop the other, whereby each vertebral body includes a relatively strong bone portion forming the outside surface of the body (cortical) and a relatively weak bone portion forming the center of the body (cancellous). Situated between each vertebral body is an intervertebral disc that provides for cushioning and dampening of compressive forces applied to the spinal column. The vertebral canal containing the delicate spinal cords and nerves is located just posterior to the vertebral bodies.

Various types of spinal column disorders are known and include scoliosis (abnormal lateral curvature of the spine), kyphosis (abnormal forward curvature of the spine, usually in the thoracic spine), excess lordosis (abnormal backward curvature of the spine, usually in the lumbar spine), spondylolisthesis (forward displacement of one vertebra over another, usually in a lumbar or cervical spine) and other disorders caused by abnormalities, disease or trauma, such as ruptured or slipped discs, degenerative disc disease, fractured vertebra, and the like. Patients that suffer from such conditions usually experience extreme and debilitating pain as well as diminished nerve function.

While the present invention can be employed for various surgical procedures it is especially advantageous for spinal fixation whereby surgical implants are used for fusing together and/or mechanically immobilizing adjacent vertebrae of the spine. Spinal fixation may also be used to alter the alignment of the adjacent vertebrae relative to one another so as to alter the overall alignment of the spine. Such techniques have been used effectively to treat the above-described conditions and, in most cases, to relieve pain suffered by the patient.

One particular spinal fixation technique includes immobilizing the spine by using orthopedic rods, commonly referred to as spine rods that run generally parallel to the spine. This is accomplished by exposing the spine posteriorly and fastening bone screws to the pedicles of the appropriate vertebrae. The pedicle screws are generally placed two per vertebra, one at each pedicle on either side of the spinous process, and serve as anchor points for the spine rods. Clamping elements adapted for receiving a spine rod therethrough are then used to join the spine rods to the screws. The aligning influence of the rods forces the spine to conform to a more desirable shape. In certain instances, the spine rods may be bent to achieve the desired curvature of the spinal column.

For example, in a procedure to fuse damaged, diseased, malformed, or otherwise abnormal vertebrae, the vertebrae are positioned in a corrected position by a surgeon. An elongated plate is placed adjacent to the vertebral bone, and bone anchors, such as specially configured screws or bolts, are employed to secure the plate to the bones. With such anchors, placement is accomplished by drilling one or more holes in the bone(s) and threading the anchors into the holes. An anchor can be connected to the bone, as by threading into a vertebral hole, through a plate, or alternatively the plate can be placed in position over or around the anchor after the anchor is connected to the bone. The anchor and plate are then secured to each other to minimize or prevent relative movement. In this way, bones may be held and/or supported in proper alignment for healing.

While various types of bone screws are known there is yet a need for surgical screw implants with improved gripping of the surrounding bone tissue to ensure a secure and permanent implantation in the bone.

### SUMMARY OF THE INVENTION

Accordingly, an implant and method are provided herein. The implant comprises an elongate body and an expander. The elongate body includes an axial bore having a bore diameter, a distal end, a proximal end, at least two longitudinal slots defining at least two expandable portions of the body in the vicinity of the distal end thereof, a threaded exterior surface and means for attachment to a lateral brace. The expander is disposed at least partially within the axial bore of the body, and includes a longitudinal shaft and a tip at the distal end of the shaft. The expander is longitudinally movable between a distal position and a proximal position, such that the expandable portions of the body are moved from an unexpanded configuration to a radially outward expanded configuration in response to movement of the expander from the distal position to the proximal position.

In an embodiment the distal end of the body includes an annular beveled surface delimited by an outer beveled surface diameter and an inner beveled surface diameter.

In an embodiment when the expander is in the distal position the tip is positioned distal to the distal end of the body and when the expander is in the proximal position at least a portion of the tip is proximal to the annular beveled surface at the distal end of the body.

In an embodiment the tip includes a distal conical portion and a proximal circumferential edge having a circumferential edge diameter greater than the inner beveled surface diameter but less than the outer beveled surface diameter.

In an embodiment the radially expandable portions of the body are movable from a first unexpanded configuration to a radially outward expanded second position in response to the camming contact of the proximal circumferential edge of the tip with the beveled surface of the body.

### BRIEF DESCRIPTION OF THE DRAWINGS

Various embodiments of the invention are described below with reference to the drawings, wherein:

FIG. 1 illustrates the fixation system of the invention;

FIGS. 2 and 3 are, respectively, side and end views of the pedicle screw of the invention in an unexpanded configuration;

FIGS. 4 and 5 are, respectively, side and end views of the pedicle screw of the invention in an expanded configuration;

FIG. 6 is a sectional view of the pedicle screw in an unexpanded configuration;

FIG. 7 is a sectional view of the pedicle screw and brace in an expended configuration;

FIG. 8 is a sectional view of an alternative embodiment of the pedicle screw of the invention;

FIG. 9 is a sectional view of the embodiment of FIG. 8 and brace in the expanded configuration; and,

FIG. 10 is a sectional view of an alternative embodiment of the invention.

### DETAILED DESCRIPTION

The present invention may be understood more readily by reference to the following detailed description of the invention taken in connection with the accompanying figures, which form a part of this disclosure and in which like numbers indicate like features. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value.

When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”. As used herein, “comprising”, containing”, “characterized by” and grammatical equivalents thereof are inclusive or open-ended terms that do not exclude additional, unrecited elements or method steps, but will also be understood to include the more restrictive terms “consisting of” and “consisting essentially of.”

The following discussion includes a description of an implant system, related components and exemplary methods of employing the implant system in accordance with the principles of the present disclosure. Reference will now be made in detail to the exemplary embodiments of the present disclosure, which are illustrated in the accompanying figures.

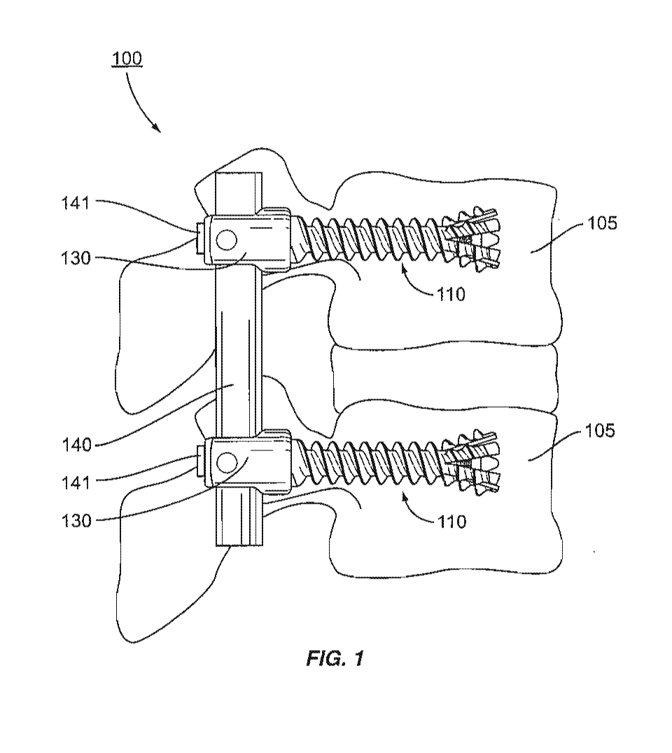

Referring now to FIG. 1-5, the expandable implant of the invention is described with reference to a spinal fixation system 100 including one or more pedicle screws 110 connected to a brace 140 for implantation into, and support of, vertebrae 105. However, as mentioned above, the fixation system can be used in any appropriate surgical procedure such as, for example, to secure pieces of broken bone(s), for ligament repair, and any other such procedures requiring surgical fixation to hard body tissues.

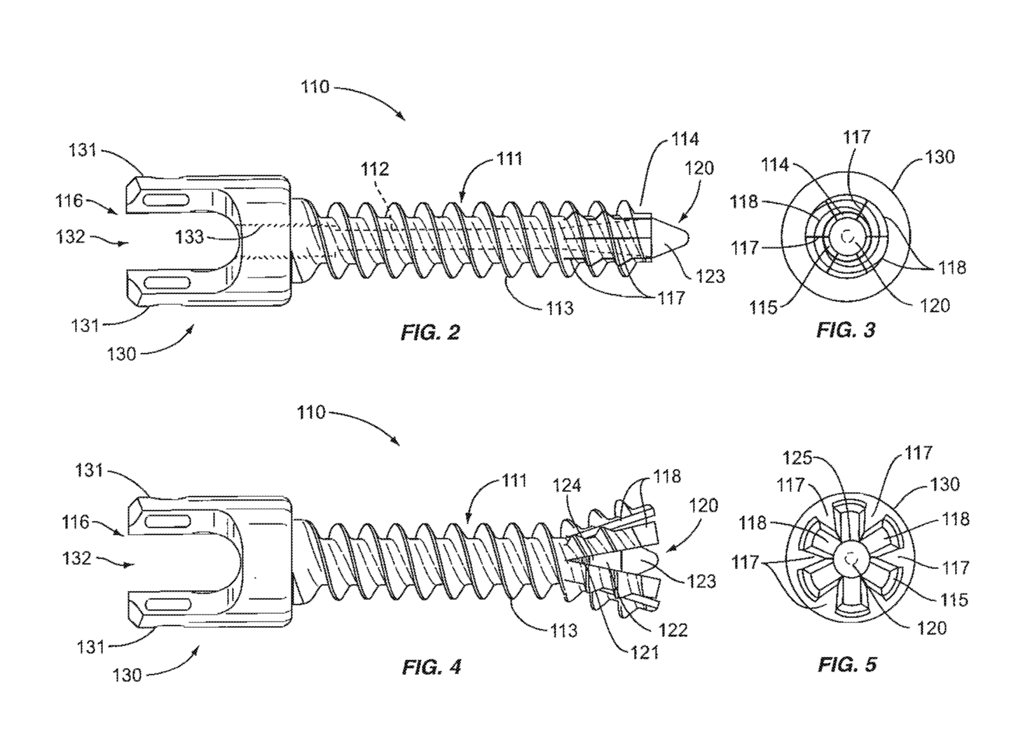

Referring now more particularly to FIGS. 2, 3 and 6, an individual expandable pedicle screw 110 is illustrated in an initial unexpanded configuration. Pedicle screw implant 110 includes a generally cylindrical elongated body 111 having an axial bore 112 and a threaded outer surface 113. In an embodiment the distal end 114 of pedicle screw 110 includes an annular beveled surface 115 delimited by an outer beveled surface diameter and an inner beveled surface diameter. In an embodiment the inner beveled surface diameter is the diameter of the axial bore 112 at the distal end 114 of the pedicle screw. In an embodiment the outer beveled surface diameter is the outer diameter of the body 111 at the distal end 114 of the pedicle screw. Optionally, the axial bore diameter can remain unchanged over the length of the bore, or can be greater or lesser at the distal end 114 of the pedicle screw than at the opposite proximal end 116. Optionally the outer diameter of the body 111 can remain unchanged over the length of the body or can be greater or lesser at the distal end 114 of the body than at the proximal end 116.

At least two, and preferably three to six, longitudinal expansion slots 117 are positioned in the vicinity of the distal end 114 of the pedicle screw 110. Expansion slots 117 define expandable members 118 which are flexibly movable from an initial unexpanded configuration, as shown in FIGS. 2 and 3, and an expanded configuration, as shown in FIGS. 4, 5 and 7, as discussed below.

Pedicle screw 110 further includes a head 130 providing means for attachment to a lateral brace. In an optional embodiment head 130 is a forked head having spaced apart posts 131 defining a gap 132 therebetween. The gap 132 is sized so as to accommodate brace 140 as described below with reference to FIG. 1. In an exemplary embodiment, the forked head 130 is of integral one-piece construction with body 111 such that axial bore 112 extends also through forked head 130. Although other head constructs and body element attributes (e.g., multiaxial head assemblies and/or thread angles) may include features found in the following references: U.S. Pat. No. 6,113,610; U.S. Pat. No. 7,291,150; U.S. Pat. No. 6,565,572; U.S. Pat. No. 6,471,703; U.S. Pat. No. 6,379,357; U.S. Pat. No. 7,699,873; U.S. Patent Publication 20080114457; U.S. Patent Publication 20070270859; U.S. Patent Publication 20090125072; U.S. Patent Publication 20080221688; U.S. Patent Publication 20100023063; International Patent Publication WO 08/014,069; International Patent Publication WO 09/108,618; International Patent Publication WO 07/087,628; International Patent Publication WO 96/32071; International Patent Publication WO 07/118,045; International Patent Publication WO 06/110796; International Patent Publication WO 09/091,686; International Patent Publication WO 08/051,737; International Patent Publication WO 10/045,453; International Patent Publication WO 98/30161; International Patent Publication WO 98/12976; International Patent Publication WO 98/34554; International Patent Publication WO 08/027,853; International Patent Publication WO 04/080318; and International Patent Publication WO 09/005,518, the entirety of each of which is incorporated by reference.

Pedicle screw 110 includes an expander 120 which is disposed at least partially within axial bore 112 and includes a longitudinal shaft 121 and a distal tip 122. The distal tip 122 includes a conical distal surface 123 and a proximal surface 124 defined by circumferential edge 125.

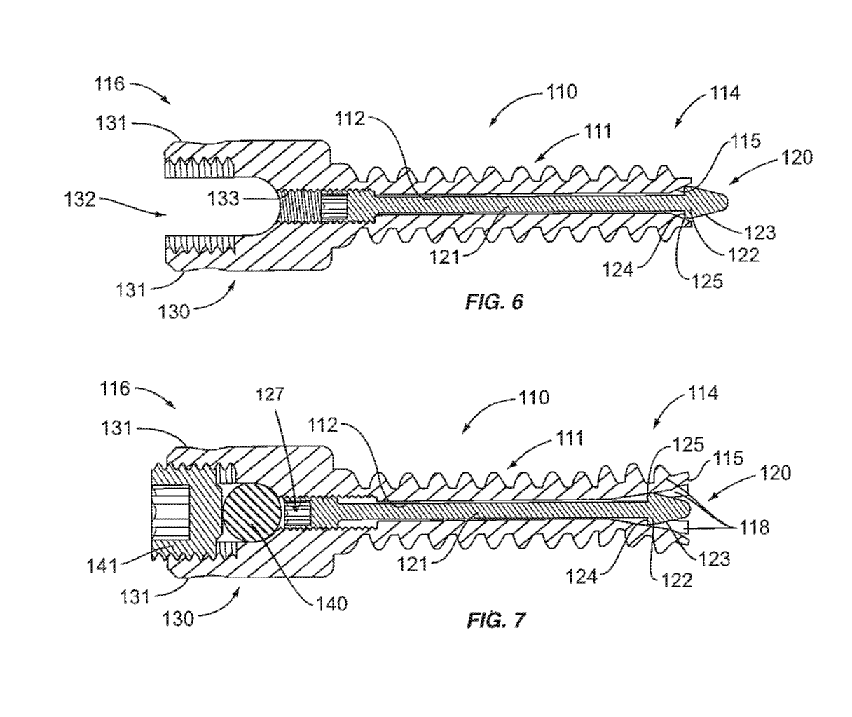

Referring to FIGS. 6 and 7, in an embodiment of the invention proximal surface 124 is perpendicular to the longitudinal axis of the body 110. Circumferential edge 125 has a diameter greater than the inner diameter of the annular beveled surface 115 and less than the outer diameter of the annular beveled surface 115. The expander 120 is longitudinally movable within body 110 between an initial distal position and a proximal position. In the distal position the tip 122 is distal to the distal end 114 of the body 111. However, as shown in FIGS. 6 and 7, as the expander 120 is moved proximally in one embodiment, the proximal edge 125 of proximal surface 124 of the tip 122 cams against the beveled surface 115 thereby forcing the expandable members 118 radially outward from an initial unexpanded configuration as shown in FIG. 2 to an expanded configuration as shown in FIG. 4. Movement of the expander can be accomplished by pulling longitudinal shaft 121 proximally. Alternatively, as shown in FIGS. 6 and 7, the proximal end portion of shaft 121 can be threaded and disposed through a corresponding axial threaded bore 133 in the forked head 130. The shaft and tip are preferably of integral single piece construction or are at least fixedly attached and immovable with respect to each other. Accordingly, longitudinal movement of the expander 120 can be accomplished by rotating shaft 121 by means of a wrench or other tool which engages the proximal end of shaft 121, for example, by engagement with a corresponding aperture 127 or other means of engagement with a tool for rotation.

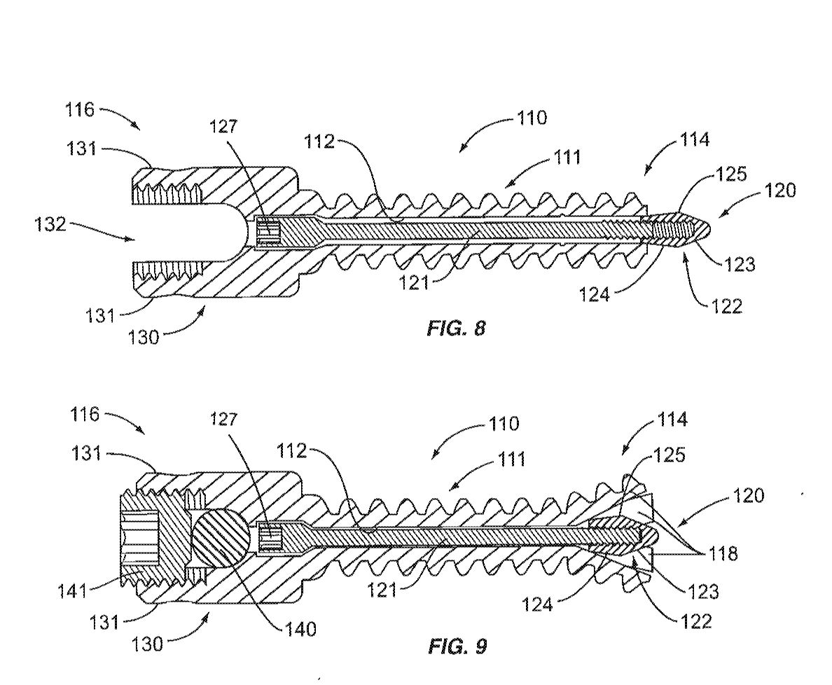

Referring to FIGS. 8 and 9, in another embodiment the distal end 114 of the pedicle screw does not have an annular beveled surface. Rather, the distal end has an annular surface that is perpendicular to the longitudinal axis of body 110 and is delimited by an inner edge having the diameter of the axial bore 112 and an outer edge having the diameter of the outer surface 113 of the pedicle screw 110. In this embodiment the proximal surface 124 of the tip 122 is beveled. The conical distal surface 123 is sized and shaped to frictionally engage a corresponding end of the hole drilled in the bone such that tip 122 does not rotate. The surface 123 can be roughened rather than smooth to facilitate frictional engagement. In this embodiment the distal end 114 of the body 111 is cammed by the beveled proximal surface 124 of the tip 122. The tip 122 is connected to shaft 121 by a threaded engagement to permit relative rotation therebetween as explained below.

Movement of the expander can be accomplished by rotating shaft 121, for example, by means of a wrench or other tool, which engages the proximal end of shaft 121, for example, by engagement with a corresponding aperture 127 or other means of engagement with a tool for rotation. As the tip 122 in this embodiment is frictionally prevented from rotation, the rotation of the shaft causes the position of the tip 122 relative to the shaft 122 and the body 111 to longitudinally change from a position relatively distal to the distal end 114 of the body 111 to a position relatively proximal to the distal end 114 of the body 111 such that a camming action is achieved between beveled proximal surface 124 and the distal end 114 of the body to radially move the expandable members 118.

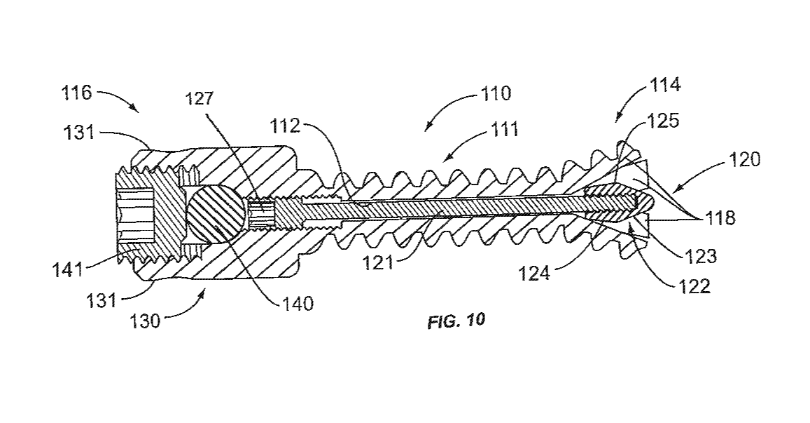

FIG. 10 illustrates an embodiment similar to that of FIGS. 8 and 9 in that the proximal surface 124 of the tip 122 is beveled. But the embodiment of FIG. 10 differs from that of the embodiment of FIGS. 8 and 9 in that, as with the embodiment illustrated in FIGS. 6 and 7, the proximal end portion of shaft 121 can be threaded and disposed through a corresponding axial threaded bore 133 in the forked head 130. The shaft 121 and tip 122 are preferably of integral single piece construction or are at least fixedly attached and immovable with respect to each other. Accordingly, longitudinal movement of the expander 120 can be accomplished by rotating shaft 121 by means of a wrench or other tool, which engages the proximal end of shaft 121, for example, by engagement with a corresponding aperture 127 or other means of engagement with a tool for rotation.

In use, a hole is drilled into bone where the pedicle screw is to be implanted. The diameter of the hole corresponds to the outer diameter of the body 111 such that the pedicle screw in the unexpanded configuration is screwed distally into the hole in a tight fitting engagement. Then, the expander 120 is moved proximally by, for example, rotating or pulling, to move the expandable members into the radially outward expanded configuration as shown in FIGS. 1, 4, 6, 7 and 10; or, in the embodiment of FIGS. 8 and 9, by rotating shaft 121 while tip 122 is frictionally engaged with the bone.

Referring to FIG. 1 the fixation system 100 further includes a brace 140 which can be disposed through gaps 132 in the forked heads 130 of two or more pedicle screws 110. As shown, one pedicle screw is inserted into each of two adjacent vertebrae 105 and moved to the expanded configuration to secure the pedicle screws 110 to the bone. Each pedicle screw is secured at the proximal end to the brace 140 by fasteners 141 which can be screw fasteners or bolts disposed through corresponding lateral apertures in the brace 140, or any other suitable method for fixation.

The brace 140 can be a rod or tube. It can have a circular cross section or polygonal cross section (e.g. square, rectangular, etc.). It can be rectilinear, arcuate, S-shaped or of some other configuration. Although only two pedicle screws are shown attached to brace 140, the fixation system can include three or more pedicle screws attached to brace 140.

The components of the fixation system may be fabricated from materials suitable for medical applications, including metals, synthetic polymers, ceramics, bone, biocompatible materials and/or their composites, depending on the particular application and/or preference of a medical practitioner. For example, components of the fixation system, such as, for example, a pedicle screw, an outer surface of the pedicle screw and/or portions thereof, cavities of the pedicle screw, which may be monolithically formed, integrally connected or configured as an insert with the body, fastening elements and/or instruments and/or expanding devices, discussed herein, can be fabricated from materials such as commercially pure titanium, titanium alloys, Grade 5 titanium, super-elastic titanium alloys, cobalt-chrome alloys, stainless steel alloys, superelastic metallic alloys (e.g. Nitinol, super elasto-plastic metals, such as GUM METAL® manufactured by Toyota Material Incorporated of Japan), thermoplastics such as polyaryletherketone (PAEK) including polyetheretherketone (PEEK), polyetherketoneketone (PEKK) and polyetherketone (PEK), carbon fiber reinforced PEEK composites, PEEK-BaSO₄ composites, ceramics and composites thereof such as calcium phosphate (e.g. SKELITE™), rigid polymers including polyphenylene, polyamide, polyimide, polyetherimide, polyethylene, polyurethanes of any durometer, epoxy and/or silicone. Different components of the fixation system may have alternative material composites to achieve various desired characteristics such as strength, rigidity, elasticity, compliance, biomechanical performance, durability and radiolucency or imaging preference. The components of the fixation system may also be fabricated from a heterogeneous material such as a combination of two or more of the above-described materials.

While the above description contains many specifics, these specifics should not be construed as limitations of the invention, but merely as exemplifications of preferred embodiments thereof. Those skilled in the art will envision many other embodiments within the scope and spirit of the invention as defined by the claims appended hereto.

What is claimed is:

1. An implant which comprises:
a) an elongated body including an axial bore having a bore diameter, a distal end, a proximal end, at least two longitudinal slots defining at least two expandable portions of the body in the vicinity of the distal end thereof, a threaded exterior surface and a head configured to engage a lateral brace;
b) an expander disposed at least partially within the axial bore of the body, said expander including a longitudinal shaft and a tip at the distal end of the shaft, said expander tip being relatively longitudinally movable between a distal position and a proximal position, wherein said expandable portions of the body are moved from an unexpanded configuration to a radially outward expanded configuration in response to relative movement of the expander tip from the distal position to the proximal position.

2. The implant of claim 1, wherein said distal end of the body includes an annular beveled surface delimited by an outer beveled surface diameter and an inner beveled surface diameter.

3. The implant of claim 2, wherein when the expander tip is in the distal position the expander tip is positioned distal to the distal end of the body and when the expander is in the proximal position at least a portion of the tip is proximal to the annular beveled surface at the distal end of the body.

4. The implant of claim 3, wherein the tip includes distal conical portion and a proximal circumferential edge having a circumferential edge diameter greater than the inner beveled surface diameter but less than the outer beveled surface diameter.

5. The implant of claim 4, wherein the radially expandable portions of the body are movable from a first unexpanded configuration to a radially outward expanded second position in response to the camming contact of the proximal circumferential edge of the tip with the beveled surface of the body.

6. The implant of claim 1, wherein the bore diameter remains unchanged from the proximal end to the distal end of the elongate body.

7. The implant of claim 1, wherein the bore diameter is greater or lesser at the distal end of the body than at the proximal end of the body.

8. The implant of claim 1, wherein the expander tip includes a proximal beveled surface.

9. The implant of claim 1, wherein the head configured to engage a lateral brace is a forked head having two spaced apart posts defining a gap therebetween disposed at disposed at the proximal end of the body.

10. The implant of claim 9, wherein the shaft of the expander includes a proximal threaded portion disposed through a corresponding threaded bore in the forked head.

11. A fixation system which comprises:
(a) at least a first implant including
(i) at least one elongate body including an axial bore having a bore diameter, a distal end, a proximal end, at least two longitudinal slots defining at least two expandable portions of the body in the vicinity of the distal end thereof, said proximal end including a head configured to engage a brace;
(ii) an expander disposed at least partially within the axial bore of the body, said expander including a longitudinal shaft and a tip at the distal end of the shaft, said expander being longitudinally movable between a distal position and a proximal position, wherein said expandable portions of the body are moved from an unexpanded configuration to a radially outward expanded configuration in response to movement of the expander from the distal position to the proximal position; and
(b) a brace connected to the head of the implant.

12. The fixation system of claim 11 including at least two implants each being connected to the brace.

13. The fixation system of claim 11, wherein the distal end of the body includes an annular beveled surface delimited by an outer beveled surface diameter and an inner beveled surface diameter.

14. The fixation system of claim 13, wherein when the expander is in the distal position the tip is positioned distal to the distal end of the body and when the expander is in the proximal position at least a portion of the tip is proximal to the annular beveled surface at the distal end of the body.

15. The fixation system of claim 14, wherein the tip includes distal conical portion and a proximal circumferential edge having a circumferential edge diameter greater than the inner beveled surface diameter but less than the outer beveled surface diameter.

16. The fixation system of claim 15, the radially expandable portions of the body are movable from a first unexpanded configuration to a radially outward expanded second position in response to the camming contact of the proximal circumferential edge of the tip with the beveled surface of the body.

17. A method for the fixation of bone tissue comprising the steps of:
a) providing the fixation system of claim 11;
b) drilling at least a first hole of predetermined size into a first portion of the bone tissue;
c) inserting the at least first implant in an unexpanded configuration distal end first into the first hole in the bone tissue;
d) moving the expander to a proximal position to move the expandable portions of the body to the expanded configuration; and
e) attaching the brace to the head of the at least first implant.

18. The method of claim 17, further comprising:
f) drilling at least a second hole in a second portion of the bone tissue;
g) inserting at least a second implant in an unexpanded configuration distal end first into the second hole in the bone tissue;
h) moving the expander to a proximal position to move the expandable portions of the body to the expanded configuration; and
i) attaching the brace to the head of the at least first implant.

19. The method of claim 18, wherein the first portion of the bone tissue is separated from the second portion of the bone tissue.

20. The method of claim 19, wherein the first and second portions of the bone tissue are individual vertebrae.
